(19) United States
(12) Patent Application Publication
(10) Pub. No.: US 2005/0149184 A1
(43) Pub. Date: Jul. 7, 2005

(54) Phakic intraocular lens with improved fluid circulation properties

(76) Inventor: Theo Bogaert, HS Groningen (NL)

Correspondence Address: DINSMORE & SHOHL, LLP, 1900 CHEMED CENTER, 255 EAST FIFTH STREET, CINCINNATI, OH, 45202, US

(21) Appl. No.: 11/011,508

(22) Filed: Dec. 14, 2004

Related U.S. Application Data
(60) Provisional application No. 60/529,538, filed on Dec. 15, 2003.

(30) Foreign Application Priority Data
Dec. 15, 2003 (SE) 0303365-1

(51) Int. Cl. A61F 2/16

(52) U.S. Cl. 623/6.14; 623/6.17; 623/6.36

(57) ABSTRACT

According to a first aspect, a phakic intraocular lens (PIOL) is suitable for implantation between the iris and the natural lens in an eye. The PIOL allows for fluid circulation between its posterior and the anterior of said natural lens after implantation. The PIOL comprises a central optic part, a peripheral haptic part, and at least one penetrating channel. The channel(s) has an anterior orifice and a posterior orifice. The channel is arranged at the border of, or outside, the central optic part. According to a second aspect, a method of preventing glaucoma associated with implantation of a phakic intraocular lens (PIOL) between the iris and the native lens in an eye comprises the step of implanting a PIOL as described.

### RELATED APPLICATION

This application claims priority under 35 U.S.C. §119 of U.S. Application Ser. No. 60/529,538 filed Dec. 15, 2003.

### FIELD OF THE INVENTION

The present invention relates to the field of implants for ophthalmic surgery. More specifically, the present invention is concerned with implantable phakic intraocular lenses, PIOLs, which are suitable as correction lenses together with the intact natural crystalline lens, or optionally an implanted crystalline lens substitute. The inventive lenses are provided with one or more penetrating channels to allow for improved fluid transport in the eye.

### BACKGROUND OF THE INVENTION

PIOLs are increasingly conceivable as an alternative to correct for optical deficiencies besides spectacles and conventional contact lenses. In a general sense, PIOLs can be considered for implantation, either in the anterior (front) chamber of the eye between the cornea and the iris, or in the posterior (rear) chamber located between the iris and the natural crystalline lens.

PIOLs positioned in the anterior chamber have been considered as desirable in several earlier embodiments for the reason that this chamber is considerably larger than the posterior chamber and thereby admitting a less complicated surgical process. However, these types of lenses show series of drawbacks essentially related with an irritation action from the support means (haptics) on the sensitive eye structures. For example, the support means can, when positioned in the corner between cornea and iris, disturb the aqueous outflow and consequently generate an increase in the intraocular pressure, a condition, which at worst may induce glaucoma. The haptic may press on the iris and disturb the blood circulation causing the pupil to aquire an oval shape. The PIOL optic in the vicinity of the cornea may contact the cornea intermittent and cause damage to the endothelium. The present invention is concerned with PIOLs to be implanted in the posterior chamber.

It is a general complication when designing PIOLs to be implanted in the posterior chamber between the iris and the natural crystalline lens that the available space is small. Consequently, the PIOLs cannot be bulky, as frequently is required when a high power optical correction is considered. Application of diffractive optics may reduce the profile of the lens, making it thinner.

In particular, consideration must be taken to avoid or restrict any contacts with the intact natural crystalline lens, in order to prevent it from damages, which may lead to local opacifications, or at worst case cataract formation.

Considerations must also be taken to that contact with posterior iris could result in abrasive intraocular damages with resulting pigment dispersion, and that the pupil must not be blocked. Blocking of the pupil prevents the flow of aqueous humor, which may lead to raised intraocular pressure and reduced circulation of nutrients and metabolites to and from the natural crystalline lens.

Various types of PIOLs are known. They could be grouped according to their design, one-piece or multiple piece PIOLs. A one-piece PIOL is one where both optic and haptic portions are made from one material. The haptic portions are used for attachment purposes. Two general designs for the haptics are a “plate-type” and a “C-haptic” type, both of which have a variety of shapes.

The preferred positioning of the PIOL is free-floating as opposed to sulcus-fixated. The PIOL will either rest on the zonula or be pushed forwards by the aqueous humor flowing from the ciliary body in anterior direction. The iris restricts the movement of the PIOL in the anterior direction. Since the PIOL is pushed forward, a distance is created between the PIOL and the crystalline lens. Thereby, the aqueous flow can reach the posterior surface of the PIOL, bring nutrients to the anterior surface of the crystalline lens, and remove products from the metabolic processes.

Since the shape of the anterior crystalline lens varies from person to person, it is not possible to avoid contact points or line contacts at all times between the PIOL and the crystalline lens. There is a risk that a line contact around the optic of the PIOL will create a sealed chamber between the central PIOL and the crystalline lens. This is a highly undesirable situation, since it will prevent nutrients to reach the central part of the PIOL and prevent derivatives of the crystalline lens metabolism from being removed. It can lead to a serious disturbance of the crystalline lens metabolism and the osmotic balance, resulting in reduced transmission of light through the crystalline lens and opacifications. The sealed chamber will also interfere with the accommodation.

When the crystalline lens accommodates, the volume of the liquid between the PIOL and the crystalline lens decreases. If this is not possible due to that the PIOL is in contact with the crystalline lens, thereby creating a sealed chamber, the accommodation will be hindered. A force will be exerted on the crystalline lens, leading to a temporary deformation of the anterior surface of the lens. A force of the same magnitude will be exerted on the PIOL, which will be pressed forward. As the force on the optic and/or optic/haptic transition zone increases, the seal thus created will improve in strength. The liquid will eventually be squeezed out of the chamber due to increased pressure, the volume of the chamber will decrease, and the crystalline lens can accommodate. In practice, there will be a mix of the two mechanisms described. The PIOL will be pressed forward, and the accommodation of the crystalline lens will to some extent be hindered. The forward movement of the PIOL can cause the anterior chamber angle to close and the risk for an increased intraocular pressure, IOP, and associated closed-angle glaucoma will increase.

If the eye changes its geometry from accommodated to relaxed state, the opposite will happen. The volume of the chamber between the PIOL and the crystalline lens will increase. If the chamber is sealed, the PIOL is sucked to the surface of the crystalline lens. The crystalline lens will deform again, and the return of the crystalline lens geometry to the relaxed state will be hindered. These periodic movements of the crystalline lens in the direction of the optical axis are also important to facilitate for the PIOL to adjust its position, i.e. center itself.

The risk for this undesired contact with the crystalline lens increases if the PIOL does not fit properly in the space between the iris and the crystalline lens. A resulting effect is that the iris is rubbing against the implant with a force. Depending on the surface characteristics of the implant, its biocompatibility and adhesion to the iris, this can cause pigment dispersion, which may lead to pigmentary glaucoma. During accommodation, the crystalline lens moves forward and increases thereby the pressure in the anterior chamber. This will cause the iris to bow posteriorly and press against the PIOL and the crystalline lens. As a result, pigment dispersion will clog the trabecular meshwork.

If the PIOL does not respect the space between the iris and the crystalline lens, the PIOL pushes the iris forwards and results in a larger contact zone between the iris and the implant. Such a situation increases the risk for pupillary block, where no aqueous fluid will be able to move between the posterior chamber and the anterior chamber via the pupil. If the pupillary block persists, it may develop into pupillary block glaucoma.

These mentioned effects could be avoided by preventing the formation of the undesired seal between the PIOL and the crystalline lens. By creating a channel between the fluid in the space that is central and posterior of the PIOL, and the anterior of the PIOL, fluid exchange at the spaces between the PIOL and the crystalline lens, and between the posterior and the anterior chamber, is secured.

These channels or holes can in theory be placed anywhere in the central part of the optic. See e.g. U.S. Pat. No. 5,480,428, which describes a corrective intraocular lens including a central, axially aligned opening that enhances liquid circulation in the eye. However, the hole scatters light, which can lead to undesired reflection images on the retina, which are experienced as glare by the end user. Moreover, the hole provides aqueous fluid from the anterior chamber, which fluid has a lower concentration of glucose than the corresponding aqueous fluid in the posterior chamber.

### SUMMARY OF THE INVENTION

It is an object of the present invention to provide a PIOL that is suitable for implantation between the iris and the native lens in an eye, and not is prone to form a sealed chamber between its posterior and the anterior of the crystalline lens.

It is another object of the present invention to provide a PIOL that allows for a suitable circulation of fluids between its posterior and the anterior of the crystalline lens.

It is one object of the present invention to provide a PIOL that allows for a suitable supply of nutrient-rich aqueous fluid to the space between the posterior of the PIOL and the anterior of the crystalline lens.

It is also an object of the present invention to provide a PIOL that prevents or decreases undesired glare phenomena experienced by its end user.

It is a further object of the present invention to provide a PIOL that prevents or decreases undesired reflection images on the retina.

For these and other objects that will be evident from the following disclosure, the present invention provides a PIOL for implantation between the iris and the natural lens in an eye, wherein the PIOL is allowing fluid circulation between its posterior and the anterior of said natural lens after implantation, comprising a central optic part, a peripheral haptic part, and at least one penetrating channel with an anterior orifice and a posterior orifice, characterized in that the channel is arranged at the border of, or outside, the central optic part.

The invention is based on the insight that the presence of such a penetrating channel outside the optic part has the advantages that formation of a sealed chamber between the PIOL and the crystalline lens and an accompanying decrease in fluid circulation are avoided, while undesired glare phenomena are prevented or decreased.

According to one aspect of the invention, the axis of symmetry of the channel intersects the optical axis of the PIOL at a point posterior to the central optic part. This configuration further improves fluid circulation in the vicinity of the PIOL.

According to an aspect of the invention, the channel is tapered towards the posterior orifice. This configuration has the advantage that light scattering by the channel is decreased, thereby avoiding or decreasing undesirable reflection images on the retina. Moreover, this setup allows for facilitated use of blunt instruments during insertion of the PIOL, which further decreases the risk for injuries during surgery. Optionally, the channel has a surface that diffuses refracted or reflected light in its anterior orifice region.

According to one aspect, the PIOL according to the invention further comprises an optic/haptic transition zone arranged between the optic part and the haptic part, which provides a smooth transition between the optic and the haptic part, thereby avoiding potential stress.

According to an aspect, the PIOL according to the invention further comprises at least one recess on its anterior side, wherein said recess is arranged outside the central optic part and is connected to the anterior orifice of the channel. This arrangement prevents the iris from blocking the orifice(s), and thereby further prevents the PIOL from sticking to the iris.

According to a preferred aspect of the invention, the area of said orifice(s) is in the range of from 0.005 to 0.4 mm², such as from 0.0125 to 0.05 mm². This area is estimated to allow for a suitable flow of aqueous fluids.

According to a first aspect of the invention, the area of each orifice is larger than 0.0003 mm². This allows for transport of cells through the channels. According to a second aspect of the invention, the area of each orifice is less than 1 μm². This prevents transport of cells through the channels.

According to a preferred aspect of the invention, the PIOL is made of a viscoelastic and oxygen-permeable material.

According to another aspect, the present invention provides a method of preventing glaucoma associated with implantation of a PIOL between the iris and the native lens in an eye, comprising the step of implanting a PIOL according to the invention. Optionally, said glaucoma is selected from pigmentary glaucoma, pupillary block glaucoma, and closed-angle glaucoma.

### BRIEF DESCRIPTION OF THE DRAWINGS

FIG. 1 is a side and a front plan view of one embodiment of the PIOL according to present invention.

FIG. 2 is a side sectional view of an eye with a human crystalline lens in an accommodated and in a not accommodated state and a PIOL according to the invention.

FIG. 3 is a side sectional view of a PIOL according to the invention and the aqueous flow anterior and posterior of the PIOL.

FIG. 4 is a front view of an alternative embodiment of the PIOL according to the invention, where the positions of the channel orifices are rotated slightly with respect to the symmetry axis of the lens.

FIG. 5 is a front view of an alternative embodiment of the PIOL according to the invention with a plurality of small channels at the periphery of and peripheral to the central optic part of the PIOL.

### DETAILED DESCRIPTION OF THE INVENTION

In the most general terms, the present invention pertains to a PIOL, i.e. an intraocular correction lens for implantation in the posterior chamber of the eye between the iris and the intact natural lens (phakic posterior chamber intraocular lenses, PPC-IOLs). The correction lens comprises a centrally located optical part, capable of providing an optical correction, and a peripherally located supporting element, or haptic part, capable of maintaining said optical part in the central location. Viewed from above at use, the correction lens will generally have a total length of from about 9 to about 13 mm and a width of from about 6 to about 8 mm. These values are confined by, and determined individually from, the size of the posterior chamber of the individual patient.

The terms “natural lens” and “crystalline lens” are used synonymously throughout this application to denote the natural accomodative lens in the eye. These terms are also intended to encompass any replacement intraocular lenses, IOLs, if present. Removal of the natural lens and implantation of such IOLs are standard procedures in cataract surgery.

The natural lens typically varies in diameter between about 9 and 10.5 mm, depending on the individual patient and his/her age. The diameter of the natural lens can be estimated as a part of the pre-surgical considerations and a suitable correction lens with a suitably extended curvature can thereby readily be selected. The curvature of the correction lens shall preferably be such that it sufficiently covers the natural lens, thereby providing for that no local pressure points are built up that can form stress concentration points or zones on the natural lens which may impair its natural metabolism and form local opacifications, which in worst case result in cataract formation and the subsequent need of surgical intervention.

The support elements preferably comprise an inner part neighboring the central optical part and an outer, peripheral part, which is designed to at least partially be in contact with the ciliary sulcus and the zonulas. According to an embodiment, the peripheral part is essentially flawlessly connected to the inner part of the support elements. Preferably, the peripheral part of the support means follows a curve that converges towards a plane perpendicular to the optical axis. This ensures that the support means are directed from the zonulas attached to the natural lens and that the corrective lens advantageously adapts to be accommodated in the free space confined by the posterior chamber of the eye between the iris and the natural lens.

According to a preferred embodiment of the present invention, the PIOLs shall be freely floating in the aqueous humor of the posterior chamber and not have any permanent engagement with ciliary sulcus constituting its inner periphery. A free floating PIOL is consequently not kept in a constant position by the ciliary sulcus, but will to a certain degree follow the eye movements, i.e. those of the natural lens during accommodation and the dilations of the pupil, while being surrounded by the aqueous humor flowing through the zonulas in anterior direction. For this reason, the PIOLs according to the present invention will preferably have a maximum diameter (including optic part and support means, i.e. haptic part) less than the average diameter of the ciliary sulcus. Suitably, the overall length of the PIOL (maximum diameter) should be about 1 mm shorter than the ciliary sulcus, or larger, to avoid excessive decentration of the PIOL from the optical axis. The overall PIOL length according to the invention is generally a compromise to obtain a floating effect while retaining a centering effect from the sulcus. Therefore, preferred PIOLs according to the invention will be centered by a combined controlled interaction with the iris and the ciliary sulcus. It is to be understood that the sulcus in practice is not circular, but rather elliptical and irregular, so a frequent touching contact between the PIOL and the sulcus will in reality be attained, which contributes to the mentioned centering effect. Should the PIOL not be sufficiently centered by the iris movements or the forces of the aqueous fluid between the PIOL and the natural lens, excessive decentration will prevented by the sulcus. For this reason and since the sulcus diameter has a tendency to shrink with increasing age of the patient, it cannot always be avoided that the overall length (maximum diameter) of the PIOL at least at some points exceeds the sulcus diameter. For PIOLs having a large diameter (above about 10.5 mm), the probability of sulcus contact increases considerably and thereby the risk of sulcus engagement that may lead to a compression of the PIOL and its axial displacement.

Favorable PIOL designs according to the invention can be found in U.S. patent application 20010051826.

In a preferred embodiment, the optical part of the PIOL is essentially circular and can be designed to correct various optical defects, including myopia and hyperopia. For example, the inventive PIOLs can be designed to correct astigmatism by designing their anterior surface toroidal or superimposing a cylindrical surface on the anterior side of the PIOL. As another example, the inventive PIOLs can correct presbyopia by applying a bi- or multifocal surface of the anterior side of the PIOL. The optically skilled person can readily apply a number of alternative anterior surfaces to provide a desired optical correction.

The size of optical part (the optical diameter) generally varies between about 4 to about 7 mm, depending on the patient and the desired optical correction.

The chamber between the central part of the PIOL and the anterior surface of the crystalline lens shall always be in contact with aqueous fluid that has a high concentration of glucose and low concentration of lactic acid. A channel in the center of the PIOL, as proposed in U.S. Pat. No. 5,480,428, will provide contact with the aqueous fluid in the anterior chamber, but this fluid has a low concentration of glucose. The distance to the source, the cilliary body, may in certain subjects be longer, especially if there is a pupillary block and the aqueous fluid flows into the anterior chamber through iridotomies. In contrast, the channel(s) according to the present invention provides direct contact with the nutrient-rich aqueous fluid of the posterior chamber of the eye.

At elder age, the pupil size will decrease. At 60 years the pupil size at night is 4.1 mm, while in bright light conditions the pupil size will be 3.1 mm. At night or during sleep, the pupil is for all ages small. The iris will rest on the anterior side of the crystalline lens or, when a PIOL is implanted, on the optic of the PIOL. To avoid light scattering by the channel(s), it is preferable to position the anterior entrance of the channels outside the optic. The anterior entrance should therefore be positioned outside, or at the outer border of, the optic zone, and preferably in the optic/haptic transition zone, such that it is in contact with the posterior chamber of the eye. The posterior entrance should be in the optic zone or in the optic/haptic transition zone, if present, such that it is in contact with the central chamber between the PIOL and the crystalline lens.

The geometry of the channels in the PIOL according to the present invention is important. Generally, the channel(s) should be as small as possible, so that the disturbance of the optical function of the PIOL is minimal.

According to one aspect of the invention, the channels should be large enough to allow passage of cells. Macrophages are the largest cells in the eye, with a typical dimension of 20 μm, corresponding to an area of approximately 0.0003 mm². It has been estimated that the aqueous flow is 2.5 ml/min. Aqueous fluid can flow with this rate from the posterior to the anterior chamber if the pupil size is 4 mm and the gap between the iris and the crystalline lens is from 1 to 2 μm. This gap corresponds to a surface of from 0.0125 to 0.0250 mm². The cross-section of the channels should be equal to this surface. If two peripheral channels are applied, their diameter should be in the range of from 0.09 to 0.125 mm.

According to another aspect of the invention, the channels should be small enough to prevent passage of cells. The diameter of the channels should be from 0.5 μm to 1 μm, corresponding to an area of approximately 1 μm². Closer to 0.5 μm is desirable, since the effect on the optical behavior due to diffraction will be minimal. If the channels have a diameter of 0.5 μm, there should be a minimal of 128 000 channels to comply with the required total flow surface. The channels should preferably have a total cross-section of 0.0125 to 0.4 mm². In this manner, a gateway has been constructed from the sulcus to the anterior chamber, independent of pupillary block. The PIOL constructed in this way can be used in the treatment of pupillary block glaucoma.

The diameter of the channels is preferably relatively small, because some resistance to the aqueous flow is desirable. During accommodation, aqueous fluid will be forced in between the implant's haptic and the crystalline lens, providing the crystalline lens with oxygen and nutrition. In this perspective, the crystalline lens is functioning as an aqueous pump.

Channels with a diameter exceeding 0.5 μm should be tapered or otherwise dimensioned to avoid directing reflected light to the fovea or focus it on other parts of the retina, which light is otherwise perceived by the patient as glare. The tapering of the channels has also the advantage that the channels can be used by the surgeon as positioning holes by using a blunt instrument smaller than the anterior entrance of the channel but larger than the posterior orifice diameter of the channel. The surface of the channel orifices may be modified so as to diffuse the refracted and reflected light. Such surface modifications include a rugged, grinded or sand blasted appearance.

The channels may be positioned near the symmetry axis in the long direction of the implant. If the line connecting the center of the channels is rotated slightly with respect to the symmetry axis, the orientation of the channel orifices can function as a reference to the surgeon for the anterior side of the PIOL. The surgeon will be able to tell by the orientation of the position holes if the PIOL is implanted in the right upside/downside orientation.

To further improve the function of the communication channels, they could be connected to one or more recesses, or indentations, on the anterior side of the PIOL. The recesses are arranged outside the central optic part and are connected to the anterior orifice of the channels. The function of the recesses is to prevent blocking of the channels by the iris, thus facilitating the flow of aqueous fluid into and out of the space between the implant and the crystalline lens. In the initial situation without the implant, the aqueous fluid in the anterior and posterior chamber flows in the direction of the sulcus when the crystalline lens accommodates. When this flow is restricted, for example by the implant, pressure builds up in the anterior chamber, pressing the iris against the implant and the zonulas, causing pupillary block and pigment dispersion, resulting in glaucoma. When posterior phakic lenses are implanted in the posterior chamber, iridotomies can be applied to compensate for the pupilary block. The iris will also in this case, due to the flow through the iridotomies, be pressed against the surface. The recesses in the PIOL will prevent the blocking of the pupil by the PIOL.

Preferably, the peripherally located indentation has a generally concave shape extending towards the inner part of the support means and the optical axis. The preferred depth of the indentations is from 0.5 to 1.25 mm. The indentations thereby form free spaces, which will both contribute to fluid circulation around the PIOL and to that the contact between the PIOL and the sulcus is restricted by these resilient peripheral members in a manner that the floating effect of the PIOL can be maintained, while the benefit of the contributory PIOL centering effect from the sulcus contact is retained.

The material of the PIOL should be highly flexible and transparent. It is preferred that the material also has viscoelastic properties. This implies that the PIOL is stiff to sudden changes and is flexible for long-term geometric changes. It should preferably be permeable to oxygen, since oxygen reaches the lens from the cornea by means of diffusion and is essential for the metabolism. The lenses according to the present invention can be made from conventional biocompatible optically clear materials of a suitable refractive index by suitable molding technologies. Depending on the material, the lenses can be molded in one singular piece (silicones or poly(methyl)methacrylate (PMMA)) or be machined by precision milling and lathe cutting (PMMA or hydrogels). The lenses can be made from stiff materials like PMMA and similar acrylates. Alternatively, the lenses can be made of a material that is foldable or compressible like polysiloxanes, hydrogels such as polyHEMA, soft acrylates and the similar. A particularly suitable polysiloxane material is described in U.S. Pat. No. 5,306,297 and a particularly suitable hydrogel is described in U.S. Pat. No. 5,717,049. The skilled person can readily conceive alternatives to these materials for the inventive correction lenses.

A suitable material for the PIOL according to the invention is a material that posses both oxygen permeability and viscoelastic properties. Examples of such materials are co-polymers of siloxane and acrylic hydrogels, often used in daily-wear contact lenses.

The corrective lenses will be described in more detail below according to specific embodiments that serve to illustrate non-limiting examples of the present invention.

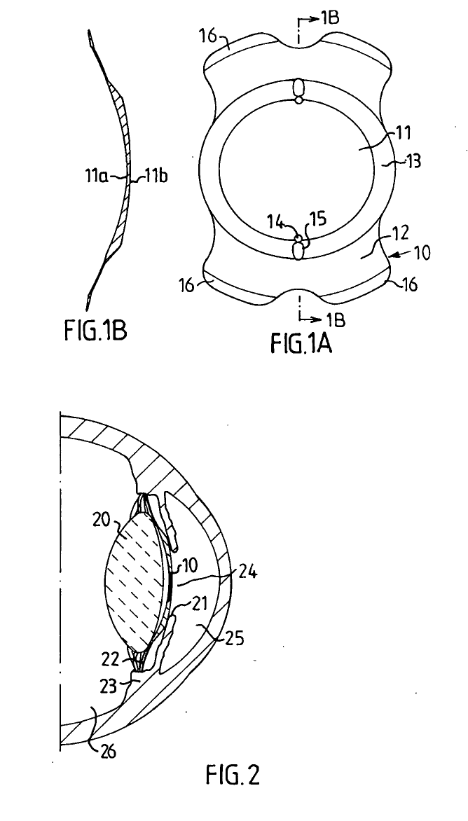

Referring to FIG. 1, there is shown a phakic intraocular lens 10 according to the present invention. The PIOL 10 includes an optic portion 11, haptic parts 12 and an optic/haptic transition zone 13. The optic portion 11 has a concave posterior surface 11a and an anterior surface 11b. The posterior surface 11a shall in use be arranged on the side corresponding to the anterior surface of the natural crystalline lens 20 (FIG. 2).

The peripherally extending recesses 15 are connected to the communication channels 14. The recesses 15 are placed outside the optical zone 11, preferrably within the optic/haptic transition zone 13.

Referring to FIG. 2, the opening in the middle of the iris 21 is the pupil 24. The chamber behind the iris is the posterior chamber 26, and the chamber in front of the iris is the anterior chamber 25. The PIOL 10 should be freely floating in the space between the crystalline lens 20 and the iris 21. This implies that the overall length of the PIOL 10 should be shorter than the diameter of the sulcus 23. To avoid excessive decentration, the overall length of the PIOL 10 should not be less than 1 mm shorter than the sulcus diameter. The sulcus 23 is however not perfectly circular. Therefore, the outer periphery of the implant should not be circular, rather be straight or having protrusions, footplates 12 (FIG. 1), in the direction of the sulcus 23. It is not necessary that the lens optic portion 11 (FIG. 1) is circular; it could also be oval, square, or any other shape as desired.

The intraocular lens 10 can be any type of PIOL, one-piece or multiple pieces IOL. The diameter of the optic portion 11 is limited within the space available. It should be large enough to avoid edge glare, but not larger, in order to minimize disturbance of the aqueous flow. The zonular free diameter is 6.86 mm. At this point the posterior radius of the PIOL 10 should increase considerable to avoid intrusion of the zonulas 22. The optic diameter should preferably not be longer than 6.5 mm. Outside a 7 mm radius, the PIOL 10 should have a thin profile in order to reduce the stiffness of the implant. An average pupil diameter is 5.1 mm at 15 Lumen. This corresponds with 4.5 mm real pupil size. The minimum optic diameter is therefore preferably set to 4.5 mm. The zonula free diameter shows variation between eyes. The design of the PIOL 10 should be robust to this. A solution is to make the design flexible. This implies a material with a lower modulus of elasticity or thinner haptics. The best option is to have the haptics thin and flexible at a diameter equal to the zonula free diameter or above.

Referring to FIGS. 1 and 2, the PIOL 10 has two communication channels 14 for the aqueous flow, positioned at the periphery of or outside the optic portion 11. To avoid blocking of the communication channels 14 by the iris 21, the anterior entrance of the communication channels 14 are positioned outside the optical portion 11. The posterior entrances of the communication channels 14 should be in the optic portion 11, or within the optic/haptic transition zone 13 (FIG. 1), and in contact with the central chamber between the PIOL 10 and the crystalline lens 20 (FIG. 2).

The crystalline lens 20 is shown in FIG. 2. When the crystalline lens 20 accommodates, the anterior radius of the central part of the crystalline lens 20 decreases, and the anterior surface moves relatively forwards with respect to the periphery.

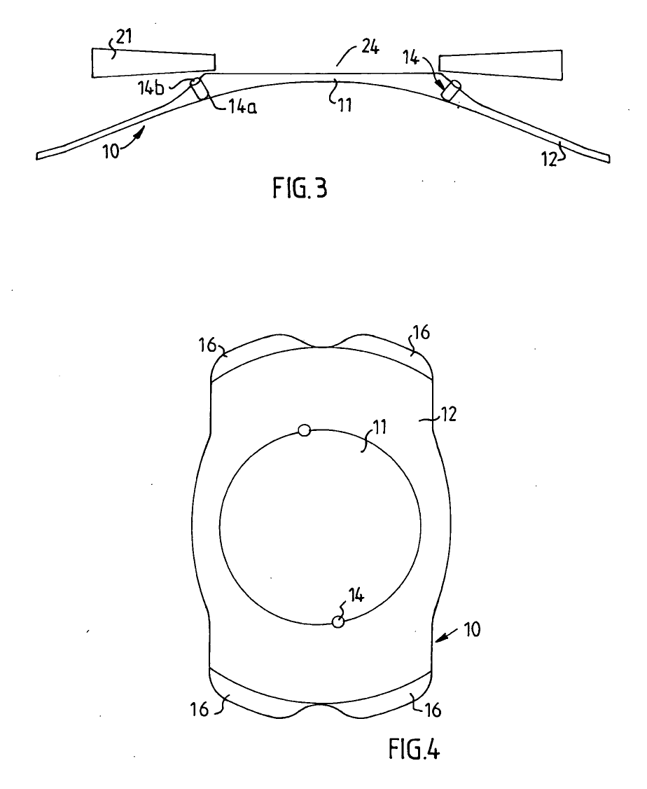

FIG. 3 illustrates an example of an intraocular lens 10 in accordance with the present invention. The anterior openings 14b of the communication channels 14 are here placed just outside the optic portion 11 of the crystalline lens 10. The posterior openings 14a are in the optic portion 11. These channels 14 will guarantee proper circulation of liquid in the eye, from the sulcus 23 (FIG. 2) into the central area posterior to the intraocular lens 10.

The communication channels 14 could be of any desired shape, straight or tapered. Communication channels 14 that are tapered towards the posterior orifice 14a have the capability to avoid scattering of incident light, which is perceived by the patient as glare. The tapering of the communication channels 14 also has the advantage that they can be used by the surgeon as positioning holes by using a blunt instrument that is smaller than the anterior entrance 14b of the communication channel 14 but larger than the posterior entrance 14a.

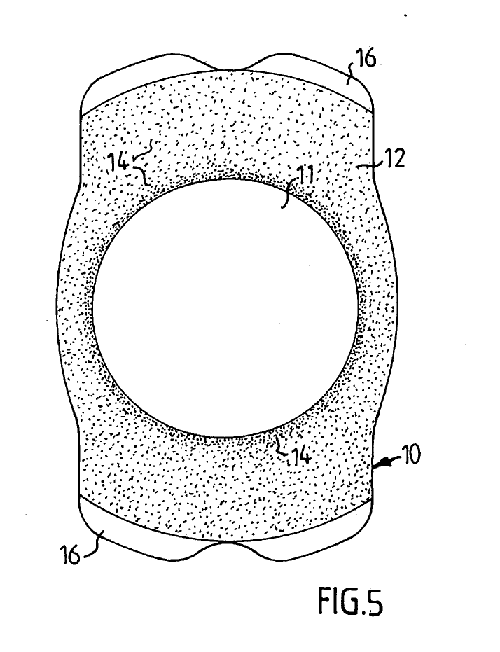

The communication channels 14 may be positioned near the symmetry axis in the long direction of the PIOL 10, as can be seen in the cross-sectional views of FIGS. 1 and 4. If the line connecting the center of the communication channels 14 is rotated slightly with respect to the symmetry axis, the orientation of the communication channels 14 can function as a reference to the surgeon for the anterior side of the PIOL 10. The surgeon will be able to tell by the orientation of the communication channels 14 if the PIOL 10 is implanted in the right upside/downside orientation.

FIG. 5 illustrates an example of a PIOL 10 according to the invention with a large number of small communication channels 14.

1. A phakic intraocular lens (PIOL) for implantation between the iris and the native lens in an eye, said PIOL allowing fluid circulation between its posterior and the anterior of said natural lens after implantation, comprising a central optic part, a peripheral haptic part, and at least one penetrating channel with an anterior orifice and a posterior orifice, wherein the channel is arranged at the border of, or outside, said central optic part.

2. A PIOL according to claim 1, wherein the axis of symmetry of said channel intersects the optical axis of said PIOL at a point posterior to said central optic part.

3. A PIOL according to claim 1, wherein said channel is tapered towards said posterior orifice.

4. A PIOL according to claim 1, wherein said channel has a surface that diffuses refracted or reflected light in its anterior orifice region.

5. A PIOL according to claim 1, further comprising an optic/haptic transition zone arranged between said optic part and said haptic part.

6. A PIOL according to claim 1, further comprising at least one recess on its anterior side, wherein said recess is arranged outside said central optic part and is connected to said anterior orifice of said channel.

7. A PIOL according to claim 1, wherein the area of said orifice(s) is in the range of from 0.005 to 0.4 mm².

8. A PIOL according to claim 7, wherein the area of said orifice(s) is in the range of from 0.0125 to 0.05 mm².

9. A PIOL according to claim 7, wherein the area of each orifice is larger than 0.0003 mm².

10. A PIOL according to claim 7, wherein the area of each orifice is less than 1 μm².

11. A PIOL according to claim 1, wherein said PIOL is made of a viscoelastic and oxygen-permeable material.

12. A method of preventing glaucoma associated with implantation of a phakic intraocular lens (PIOL) between the iris and the native lens in an eye, comprising the step of implanting a PIOL according to claim 1.

13. A method according to claim 12, wherein said glaucoma is selected from pigmentary glaucoma, pupillary block glaucoma, and closed-angle glaucoma.
